(19) United States
(12) Patent Application Publication
(10) Pub. No.: US 2016/0002710 A1
(43) Pub. Date: Jan. 7, 2016

(54) REPORTER PHAGE AND DIAGNOSTIC FOR BACTERIA

(71) Applicant: Guild Associates, Inc., Dublin, OH (US)

(72) Inventors: David A. Schofield, Hollywood, SC (US); Darren J. Wray, Charleston, SC (US); Ian J. Molineux, Bee Cave, TX (US)

(21) Appl. No.: 14/668,194

(22) Filed: Mar. 25, 2015

Related U.S. Application Data
(60) Provisional application No. 61/970,031, filed on Mar. 25, 2014.

(51) Int. Cl. C12Q 1/68

(52) U.S. Cl. CPC C12Q 1/689; C12Q 2600/158

(57) ABSTRACT

The present disclosure relates to compositions, methods, systems and kits for the detection of microorganisms of the Shigella species, including S. flexneri, S. dysenteriae, S. sonnei, and S. boydii. The disclosure relates to recombinant phage operable to infect a S. flexneri microorganism, the phage comprising a detectable reporter. Detection systems of the disclosure may comprise a phage operable to infect a S. flexneri microorganism, and may comprise a reporter nucleic acid expressible upon infection of a S. flexneri microorganism by the phage. The system may be operable to detect the expression of the reporter. A detectable reporter may comprise any gene having bioluminescent, colorimetric and/or visual detectability. Live and infectious S. flexneri microbes may be detected by the compositions, methods, systems and kits described herein.

### CROSS REFERENCE TO RELATED APPLICATIONS

The present application claims priority under 35 U.S.C. §119(e) of U.S. Patent Provisional Application Ser. No. 61/970,031, filed Mar. 25, 2014, the disclosure of which are incorporated by reference herein.

### GOVERNMENT RIGHTS

This invention was made with government support under NIH Grant Number 4R33AI100164-04, titled “Bioluminescent Reporter Phage for the Diagnostic Detection of Shigellosis”. The U.S. Government has certain rights in the invention.

### BACKGROUND

Acute diarrheal diseases are the second leading cause of death among infectious diseases. Of these, shigellosis, also known as bacterial dysentery, is a global human health problem. Shigellosis, caused by the genus Shigella, is a significant cause of morbidity and mortality, accounting for 164 million cases worldwide and 1.1 million deaths annually, most notably among children under 5 years old. The vast majority of infections occur in developing countries where poor sanitary conditions, contaminated food and water supplies, malnourishment, and overcrowded conditions are prevalent. However, shigellosis is also common in the U.S., accounting for approximately 450,000 cases per year. Shigellosis is highly contagious; the infectious dose has been estimated at 10-100 cells, and is usually transmitted by the fecal-oral route. Symptoms include loose stools mixed with blood and mucus, which are usually accompanied by abdominal cramps and fever. In the majority of cases, the disease is self-limiting. However, in severe cases, shigellosis is life-threatening and requires appropriate medication.

Shigella is a member of the Enterobacteriaceae. Organisms are small, non-motile, fastidious Gram-negative facultative anaerobic bacilli. The four species of Shigella are divided into a number of different serotypes: S. dysenteriae types 1-13; S. flexneri types 1-15; S. sonnei type 1, and S. boydii types 1-18. Of these, S. flexneri type 2a, and S. dysenteriae type 1 are responsible for the majority and the most severe infections, respectively.

Mucus and blood in stool samples are typical features of bacterial dysentery; however, the diarrheal stage of infection cannot be distinguished clinically from other bacterial, viral, and protozoan infections. Presumptive identification of Shigella infection can be made by culturing bacterial samples onto semi-selective medium, e.g., MacConkey or deoxycholate citrate agar, or highly selective media such as xylose-lysin deoxycholate, hektoen enteric, or Salmonella-Shigella agar. Confirmatory identification using real-time PCR analysis can expedite detection with sensitivity limits of detection as low as 10³ CFU/g of stool. However, the cost of molecular assays can be prohibitive to their adoption, especially to developing countries where bacterial dysentery is endemic.

Due to their inherent bacterial specificity, bacteriophages (phages) have been developed as diagnostic devices, in particular as reporters, for bacterial pathogens including Mycobacterium tuberculosis, Yersinia pestis, Bacillus anthracis, Salmonella enterica, and Listeria monocytogenes. There is a need for a reporter phage assay as a diagnostic tool for detection of the leading causes of bacterial dysentery.

### BRIEF DESCRIPTION OF DRAWINGS

FIG. 1 is a schematic illustration of LuxAB genomic location according to one example of the claimed technology.

FIG. 2 shows the PCR identification of Shfl25875::luxAB according to one example of the claimed technology.

FIG. 3 shows a growth curve of S. flexneri culture infected with Shfl25875 according to one example of the claimed technology.

FIG. 4 is a chart showing the signal response time of S. flexneri culture mixed with Shfl25875::luxAB according to one example of the claimed technology.

FIG. 5 is a chart showing the sensitivity limit detection of one example of the claimed technology.

FIG. 6 is a chart showing antibiotic susceptibility profiles of S. flexneri ATCC 25875 generated with ampicillin.

FIG. 7 is a chart showing antibiotic susceptibility profiles of S. flexneri ATCC 25875 were generated with ciprofloxacin.

FIG. 8 is a chart showing reporter phage detection of S. flexneri from spiked human stool.

FIG. 9 is a chart showing detection of S. sonnei 9290 and S. flexneri 7-3510 with Sdys9750::luxAB according to another example of the claimed technology.

FIG. 10 is a chart showing detection of S. sonnei 9290 and S. flexneri 7-3510 with Sdys12039::luxAB according to still another example of the claimed technology.

### DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

The disclosed technology describes the development of a reporter phage assay as a diagnostic tool for detection of the leading cause of bacterial dysentery, S. flexneri. In one example, wastewater samples were screened for the presence of phages displaying broad host range. One phage, Shfl25875, which displayed the broadest S. flexneri tropism, was characterized by genome sequencing, and was then engineered with the genes encoding bacterial luciferase to generate a ‘light-tagged’ reporter phage. Shfl25875::luxAB rapidly transduces bioluminescence to S. flexneri with high sensitivity, thereby providing a useful diagnostic reagent. Furthermore, as shigellosis is most problematic in developing countries where health-care expenditures are extremely limited, the low cost of phage-based assays are particularly attractive.

### Bacterial Strains and Culture Conditions.

Bacterial strains were purchased from the NIH Biodefense and Emerging Infections Research Resources Repository (Shigella spp., S. enterica, L. monocytogenes, Yersinia enterocolitica, Klebsiella pneumoniae, Escherichia coli), the American Type Culture Collection (Shigella spp. and K. pneumoniae), and the Bacillus Group Stock Center (Bacillus cereus). Some Shigella isolates were from outbreaks in South America (Guatemala and Chile). Bacteria were grown in Luria-Bertani (Enterobacteriaceae) or Brain Heart Infusion (B. cereus and L. monocytogenes) media at 37° C. with aeration. Unless otherwise stated, isolated colonies were incubated in 2 mL of media for 18-24 h to generate saturated cultures. Cultures were then diluted 1:50 to 1:200 in fresh media and incubated until A600 of 0.2 (for bioluminescent assays) or 0.4 (for phage titering and host range studies). Where indicated, bacteria were enumerated by determining colony-forming units (CFU) after 18-24 h growth at 37° C.

Isolation of Shigella Phages from Environmental Sources and Phage Propagation.

Raw wastewater samples (40 mL aliquots) were processed immediately by the addition of NaCl (final 0.7 M) and CaCl₂ (final 5 mM) with mixing for 30 min at 30° C. Particulate matter and bacteria were removed by centrifugation (4,000×g, 10 min, 4° C.), and the supernatant was made 9% (w/v) polyethylene glycol 8,000 to precipitate phages. After 3 h at 4° C. with gentle mixing, the precipitate was collected by centrifugation (11,000×g, 30 min, 4° C.) and was then gently resuspended in SM buffer supplemented with 5 mM CaCl₂ before storing at 4° C.

Shigella phages were isolated using S. flexneri serotype 2a and S. dysenteriae serotype 1 as hosts using soft agar overlays. Individual plaques were picked, serially diluted in SMC buffer and thrice plaque purified to ensure clonality of the isolated phage. Phages were then amplified in liquid culture using growing cultures until lysis, and phage lysates were clarified by centrifugation and filtration.

Host range determination of phages was performed by spotting phage dilutions using Shigella spp. (73 strains), closely related Enterobacteriaceae species (39 strains), and clinically relevant non-Enterobacteriaceae (10 strains).

### Phage DNA Sequencing.

Phage DNA was prepared and sequencing was performed by the Medical University of South Carolina Proteogenomics Facility using Ion Torrent Sequencing. The genome sequence of Shfl25875 is found at GenBank KM407600.

Construction and Generation of Recombinant Shfl::luxAB Reporter Phage.

Vibrio harveyi luxA and luxB genes were used as the reporter and were targeted for integration downstream of, and in the same orientation as gene 32 at position 146,610 by within the phage genome (FIG. 1). LuxAB was targeted for integration into the phage genome using homologous recombination. Recombinant luxAB-phage were screened and selected for based on the ability of infected cultures to acquire a bioluminescent phenotype.

### Recombinant Phage Verification.

To identify the presence of luxAB, and to confirm that integration had occurred at the correct site, cell-free phage supernatants were analyzed by PCR. Internal primers were designed to detect luxB (5′ primer ATCGACCAACGGATTCTCAG; 3′ primer ACTTCTTTGCTCGTCGCATT, product size of 184 bp). Primers were also designed to span the 5′ and 3′ integration junction sites (5′: 5′ primer CTTGTCCGTTTGAAGGTGCT; 3′ primer GCTTTGCCCAGATTAACCAA, 511 bp product: 3′: 5′ primer AGCTCGCGTGTATTTGGAAG; 3′ primer ACCACCGGCAGAACATACAG, 573 bp product). Each primer set was designed to ensure that primer binding occurred both inside and outside the original integration cassette. PCR analysis was performed as recommended by the Taq DNA polymerase manufacturer (New England Biolabs).

### Antibiotic Susceptibility Assays.

The ability of the reporter phage to confer a bioluminescent signal to S. flexneri in the presence of ciprofloxacin or ampicillin (Sigma Aldrich) was compared to the Clinical Laboratory Standards Institute (CLSI) broth microdilution method. Cells (5×10⁵ CFU/mL) and antibiotics were prepared in cation-adjusted Mueller Hinton broth according to CLSI methodology. Cells were incubated with ampicillin (0.06 to 8 μg/mL) or ciprofloxacin (0.0005 to 0.12 μg/mL) in microtiter plates, incubated at 35° C., and assessed for growth (A₆₂₅) after 20 h as per the CLSI protocol. The MIC using the internal QC strain E. coli ATCC 25922 for ampicillin and ciprofloxacin was 2 and 0.015 μg/mL, respectively, both within the acceptable range. The same Shigella inoculum was grown for 5 h at 35° C. in the presence of antibiotics, infected with Shfl::luxAB, and bioluminescence assayed after 20 min.

Detection of S. flexneri in Spiked Stool Samples.

Human stool samples from healthy individuals were sterilized by autoclaving, and spiked (10 μL) with S. flexneri (10³-10⁶ CFU/g). Stool samples (n=3 each for both the no-cell control and spiked samples) were processed by mixing with 9 mL of LB, vortexing vigorously for 5 s, and incubating at 37° C. with aeration. After 4 h, aliquots were infected with Shfl25875::luxAB and analyzed for bioluminescence.

### Bioluminescence Assays.

Unless otherwise stated, Shfl25875::luxAB (3×10⁸ PFU/mL final) and cells were mixed and incubated at the designated temperatures for set times. ‘Flash’ bioluminescence was measured using a luminometer. Cultures (195 μl per reading) were injected with n-decanal and read. Controls consisted of both cells alone and phage alone. Bioluminescence is depicted as relative light units (RLU) and the data presented are the average of three experiments SD unless otherwise stated. Statistical significance was determined using Student's t-test (p<0.05).

Isolation of Phages with a Broad S. flexneri Tropism.

S. flexneri serotype 2a is responsible for the majority of shigellosis worldwide. In addition, the occurrence of drug-resistant isolates for the epidemic S. dysenteriae serotype 1 is increasingly common. Phages were therefore isolated using S. flexneri serotype 2a, and S. dysenteriae serotype 1 as hosts. Over 100 phages displaying differences in plaque morphology (size, turbidity, clarity), titer, and stability were isolated and were then screened for host range against various S. flexneri serotypes and S. dysenteriae serotype 1. The vast majority of phages displayed a narrow host range; able to grow on the serotype used for isolation, but exhibited a reduced efficiency of plating (eop) on other serotypes (data not shown). However, one phage (named Shfl25875) grew on 28/29 of S. flexneri and all twelve S. dysenteriae type 1 strains with an eop of >0.1, and displayed an inability to grow on non-Shigella spp. (Table 1, and data not shown). Shfl25875 produces clear ˜3 mm plaques on S. flexneri ATCC 25875 and normally yields titers of >10¹⁰ PFU/mL in plate stocks. No significant loss in titer was noted after storage for 8 months at 4° C. Shfl25875 was therefore selected for further characterization, including genome sequencing and reporter phage development.

TABLE 1
Shigella inclusivity host range analysis with Shfl25875

| | | Number of strains |
|---|---|---|
| Species | Serotype | infected/total tested |
| S. flexneri | 1a | 2/2 |
| S. flexneri | 1b | 1/1 |
| S. flexneri | 2a | 16/16 |
| S. flexneri | 2b | 1/1 |
| S. flexneri | 3 | 1/1 |
| S. flexneri | 4 | 3/3 |
| S. flexneri | 5 | 1/1 |
| S. flexneri | 6 | 1/2 |
| S. flexneri | Y | 1/1 |
| S. flexneri | Unknown | 1/1 |
| S. dysenteriae | Type 1 | 12/12 |

“Infected” defined by having an efficiency of plating of >0.1 relative to host strain S. flexneri

### Genome Analysis.

Assembly of the Ion-Torrent output sequence of the Shfl25875 genome generated a single contig of 169,062 bp. The closest match in GenBank is RB69 (GenBank AY303349.1), a 167,560 bp coliphage genome with which Shfl25875 shared ˜97% sequence identity. RB69 is a member of the Tevenvirinae and is thus a T4-like phage, and homologs of all known essential, and most characterized non-essential genes of RB69 are both present in and syntenic with Shfl25875. The major differences are that Shfl25875 encodes a putative internal head protein IpII, which is present in only some members of the T4-like phages, and a putative segD-like homing endonuclease, a type of element common to many T4-like phages, but completely lacking in RB69. It also contains two orfs between cd and cd.2, one of which codes for a protein of the AroG superfamily, with 69% similarity to S. dysenteriae phospho-2-dehydro-3-deoxyheptonate aldolase. Conversely, Shfl25875 lacks the RB69 protease-inhibitor gene pin. The most closely related phage whose host is described as S. flexneri is Shfl2 (GenBank HM035025.1) with ˜80% sequence identity to Shfl25875. The long tail fibers, responsible for initial Shfl25875 adsorption to S. flexneri are, however, more similar to certain T4-like coliphages than to Shfl2.

Integration of the luxAB Reporter into the Phage Genome.

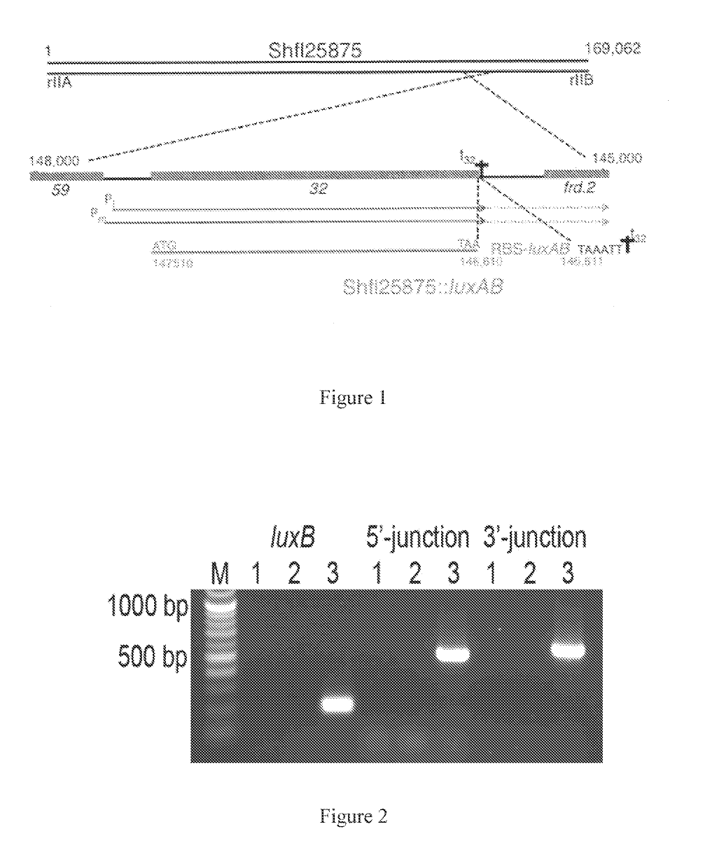

LuxAB was inserted into the phage genome by homologous recombination without deleting any phage DNA. The genome size of Shfl25875::luxAB is thus 2,108 bp greater than its parent phage Shfl25875. The inserted DNA reduces the length of the terminal redundancy associated with all T4-like phage genomes; redundancy in the T4 genome was estimated at ˜5 kb, and assuming a comparable length for Shfl25875, luxAB insertion causes a 40% reduction. However, no significant growth defect of Shfl25875::luxAB has yet been noted (see below). PCR was used to verify the presence of luxAB in the recombinant phage, and that it had integrated correctly into the targeted site (FIG. 2). PCR of phage lysates using primers designed to amplify a portion of luxB, and separately, to span the 5′ and 3′ junctions of luxAB when integrated into the phage genome, generated the expected size PCR products.

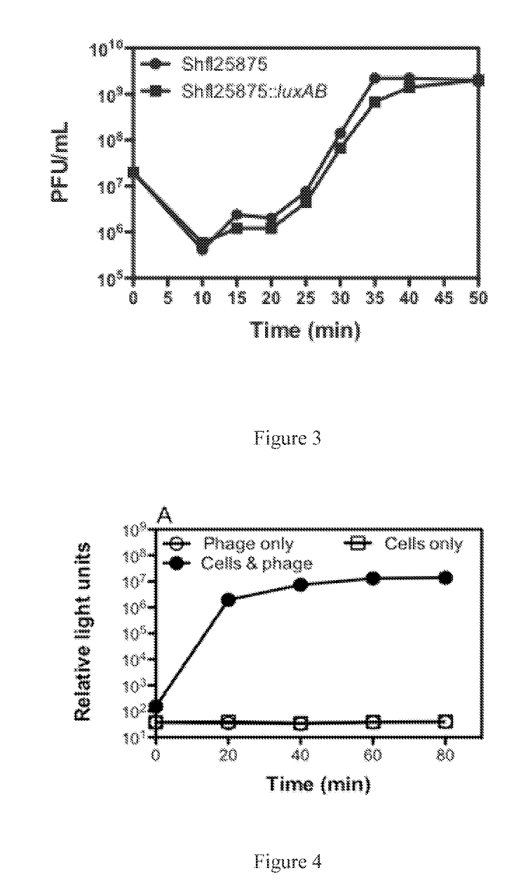

A one step growth curve compares the fitness of Shfl25875::luxAB to its parent. Both phages exhibit similar growth profiles (FIG. 3), including a 25-30 min latent period and average burst size (90 and 76, respectively for Shfl25875 and Shfl25875::luxAB) typical values for T4-like phages. These data further show that insertion of luxAB into Shfl25875 did not negatively affect fitness.

Shfl25875::luxAB-Mediated Detection of S. flexneri.

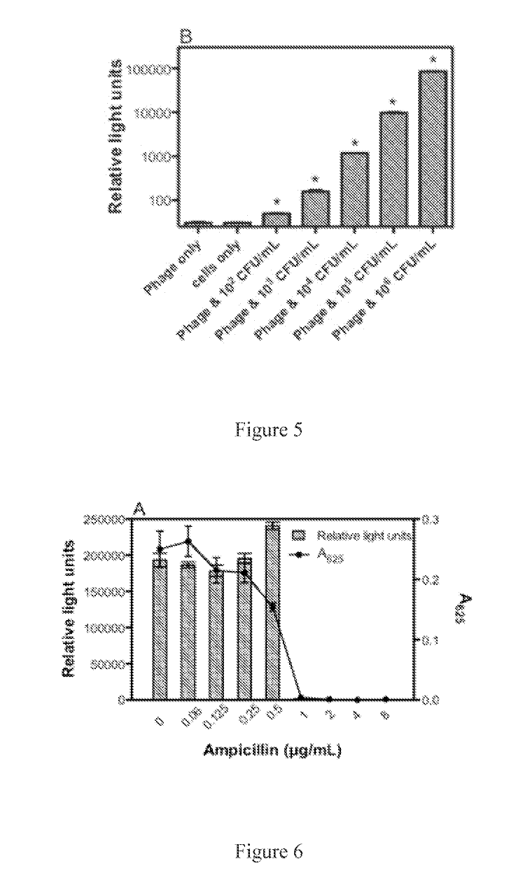

The reporter phage transduces bioluminescence to S. flexneri within 20 min of infection (FIG. 4). A significant increase in signal resulted, indicating that Shfl25875::luxAB expresses luxAB at significant levels soon after infection. The signal reaches its maximum level by approximately 60 min, and incubation for more than 120 min resulted in a drop in signal. Sensitivity limits of detection using serial dilutions of S. flexneri show dose-dependent characteristics, with increasingly higher number of cells displaying proportionally higher signals (FIG. 5). The limit of detection was approximately 10² CFU/mL within 60 min of infection (p<0.05). Collectively, the data indicate that Shfl25875::luxAB rapidly transduces a strong bioluminescent phenotype to S. flexneri in pure culture.

### Inclusivity and Specificity of the Reporter Phage.

We determined whether Shfl25875::luxAB could transduce a signal response to all four Shigella species, to closely related non-Shigella Enterobacteriaceae (E. coli, S. enterica, Y. enterocolitica, K. pneumoniae) and to more distantly related but clinically relevant enteric pathogens (B. cereus, L. monocytogenes) (Table 2). Shfl25875::luxAB detected 28/29 S. flexneri strains of different serotypes and detected 12/12 S. dysenteriae serotype 1 strains. Shfl25875::luxAB also detected 24/27 S. sonnei serotype 1 isolates. Two of 10 E. coli strains (BEI NR-3 and NR-12) elicited signals that were approximately 10- and 100-fold lower than with Shigella but only 1 of 29 strains of other Enterobacteriaceae (Y. enterocolitica, S. enterica, and K. pneumoniae) resulted in a positive signal response (S. enterica strain); this was also 100-fold lower than with the Shigella host strain. As may be expected, the distantly related species B. cereus and L. monocytogenes did not produce signals above background.

TABLE 2
Specificity of Shfl25875::luxAB phage among Shigella
species closely related species and non-related but
clinically relevant pathogens.

| | Light-positive | |
|---|---|---|
| | strains/total tested^{e} | Notes |
| Shigella spp. | | |
| S. flexneri^{a} | 28/29 | One serotype 6 strain was |
| | | negative |
| S. dysenteriae^{b} | 12/12 | All serotype 1 strains |
| S. sonnei | 24/27 | |
| S. boydii | 0/5 | Serotypes 1-5 |
| Enterobacteriaceae | | |
| E. coli^{c} | 2/10 | 2 positive strains 10 to 100- |
| | | fold lower than S. flexneri |
| K. pneumoniae^{d} | 0/10 | Background |
| S. enterica | 1/9 | 1 positive strain >100-fold |
| | | lower signal than S. flexneri |
| Y. enterocolitica | 0/10 | |
| Non- | | |
| Enterobacteriaceae | | |
| B. cereus | 0/5 | Background |
| L. monocytogenes | 0/5 | Background |

^{a}S. flexneri comprising serotypes 1a, 1b, 2a, 2b, 3, 4, 4a, 4b, 5, 6, Y
^{b}S. dysenteriae serotypes 2 through 12 only found 2 positive strains out of 12 analyzed (data not shown)
^{c}Various O-antigen strains such as O157:H7, O145:H2, O111, O121, and a uropathogenic strain
^{d}Including clinical strains isolated from stool and urine
^{e}‘Light positive strains’ defined by phage-infected strains exhibiting a bioluminescence signal response of >10³-fold over background controls

### Rapid Determination of Antibiotic Susceptibility.

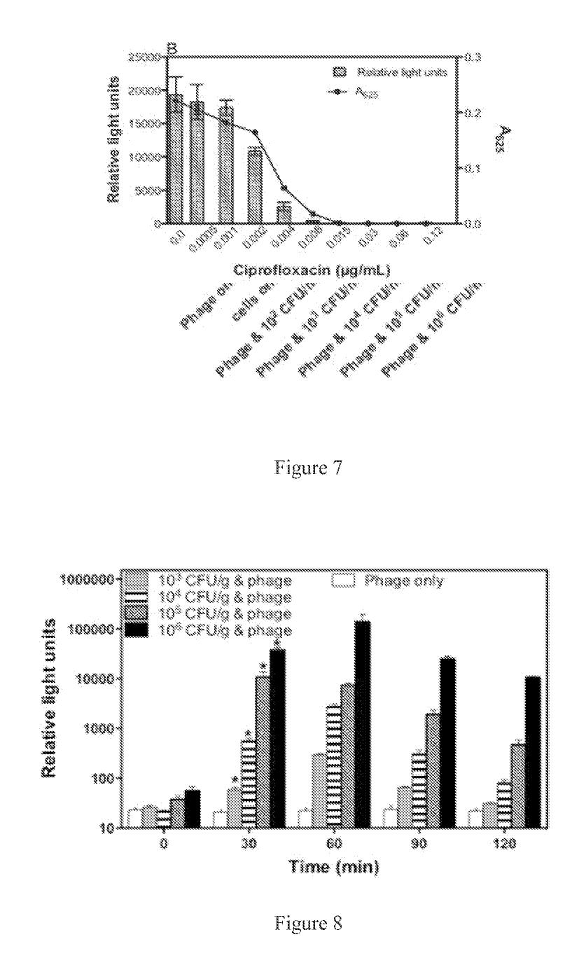

A phage-mediated bioluminescent signal response is strictly correlated to cell fitness. Therefore, the ability of Shfl25875::luxAB to confer bioluminescence signal to Shigella spp. in the presence of antibiotics was compared to the standard CLSI broth microdilution method. Standard antibiotics used for determining susceptibility of Shigella isolates include ampicillin and ciprofloxacin. Cells were incubated with a range of antibiotic concentrations in microtiter plates, incubated at 35° C., and assessed for growth (A₆₂₅) after 20 h as per the CLSI protocol, or bioluminescence following infection by Shfl25875::luxAB. The bioluminescence signal mirrored the growth profile in the presence of ampicillin or ciprofloxacin (FIGS. 6-7). At antibiotic concentrations that had little to no effect on growth, the signal from the reporter phage was near maximum. Conversely, at antibiotic concentrations that were at the minimum inhibitory concentrations (1 and 0.015 μg/mL for ampicillin and ciprofloxacin, respectively) or higher, bioluminescence was at or close to background. However, the CLSI protocol requires 16-20 h to complete while the reporter phage only requires ˜5 h. Shfl25875::luxAB not only diagnoses shigellosis but also simultaneously gathers antibiotic susceptibility data.

Phage-Mediated Detection of S. flexneri in Human Stool.

The standard clinical diagnostic specimen for bacterial dysentery is stool. Whether Shfl25875::luxAB could transduce bioluminescence to S. flexneri in deliberately spiked stool samples was tested. A discernable signal-to-noise level was observed 30 min after infection (FIG. 8). As with pure cultures, Shfl25875::luxAB exhibited typical dose-response characteristics with a sensitivity of detection of 10³ CFU/g (p<0.05). This level of sensitivity is compatible with clinical samples as Shigella spp. are shed in large numbers during the acute phase of infection.

### Overview

The isolation of Shigella phages from environmental waters in developing countries where bacterial dysentery is common has been described numerous times in the literature. The selection of the T4-like phage Shfl25875 for diagnostic development was based on its broad host range and its obligate lytic growth characteristic; Shfl25875 plaques on most S. flexneri serotypes and S. dysenteriae serotype 1 strains, but did not grow on most non-Shigella Enterobacteriaceae. That Shfl25875 infects some E. coli strains is not surprising given their very close relationship. Taxonomy places the entire Shigella genus within the species E. coli, and restriction-modification was discovered more than 60 years ago using phages that grow on both E. coli and S. dysenteriae.

In one example, Shfl25875::luxAB elicited a 10⁵-fold increase in bioluminescence to S. flexneri within 20 min. This strong response may be attributed to the position of the reporter within the phage genome. A priori, maximal expression of a phage-carried reporter gene occurs from the strongest promoters. In dsDNA phage genomes, these promoters usually direct expression of the structural genes, which code for proteins that are produced in greatest abundance in the infected cell. However, these promoters are usually activated late in infection, providing only a narrow time window for expression and function of a reporter gene construct. T4 gene 32 is transcribed throughout infection from several promoters, and its protein product acts stoichiometrically on ssDNA generated during phage DNA metabolism. Sequences corresponding to putative middle (mot-dependent) and late promoters were identified upstream of Shfl25875 gene 32, and the gene is followed by a putative terminator, as in T4. Transcriptional regulation of Shfl25875 gene 32 is comparable to that of its T4 counterpart, and thus that luxAB would be expressed at high levels if the cassette was inserted between gene 32 and its transcriptional terminator. This high level expression resulted in a sensitivity limit of detection of 300 CFU/mL in pure culture and ˜10³ CFU/g of spiked stool, suggesting that further development of Shfl25875::luxAB will result in a valuable diagnostic.

Importantly, simultaneously with diagnosis, Shfl25875::luxAB provides antibiotic susceptibility profiles because the phage uses the host's biosynthetic machinery to elicit bioluminescence. Although empiric antibiotic treatment has traditionally been the routine for shigellosis, this strategy is increasingly problematic due to antibiotic-resistant strains, the epidemic and pandemic S. dysenteriae serotype 1 strain in particular. Resistance to ampicillin, tetracycline, and nalidixic acid and other fluoroquinolones has been observed in various regions of the world. As CLSI protocols for determining antibiotic susceptibility requires 16-20 h, bioluminescence conferred by Shfl25875::luxAB significantly speeds up analysis. A similar strategy has been employed for the identification and drug susceptibility testing of M. tuberculosis isolates using recombinant mycobacteriophages.

There are currently 2 FDA-approved/cleared phage-based diagnostic assays, both of which use wild-type phage and are based on phage amplification; the phage lysis assay for B. anthracis and KeyPath™ Blood Culture Test for identifying Staphylococcus aureus and differentiating MRSA and MSSA.

### Alternative Insertion Location.

Targeted insertions between the scaffolding protein gene and major capsid protein gene were also attempted. The rationale is that the major capsid protein is the most abundant phage protein made after infection, and thus that mRNA levels in this region of the genome are also likely high. In a recombinant phage, luxAB are thus also to be expected to be highly transcribed. Inserted DNA fragments included a duplication of the late promoter sequence (TATAAATA), in order to ensure adequate transcription of the downstream gene 23; the late promoter and the complete natural intergenic sequence between genes 22 and 23, in case that short sequence was important for gene 23 expression. PCR of DNA found in lysates indicated that the expected recombination between the phage and the luxAB plasmids had occurred but luminescent phages were not found. As adequate numbers of plaques were screened, the reason for this failed attempt is unknown but may be hypothesized that the luxAB insert was deleterious because the amounts of gp22 (scaffold) and gp23 (capsid) actually synthesized in a recombinant phage was imbalanced, causing interference with the phage assembly process.

### Alternative Phages Sdys9752 and Sdys12039

The previous examples isolated, characterized, and genetically engineered a Shigella phage named Shfl25875 which could be used for the detection of certain Shigella bacterial strains. The Shigella genus is classified by four serogroups: S. dysenteriae (15 serotypes); S. flexneri (six serotypes); S. boydii (19 serotypes); S. sonnei (1 serotype). While the Shfl25875 phage infected many Shigella serogroups, it did not infect them all. Two additional phages, named Sdys9752 and Sdys12039, were also developed using similar techniques to those previously described with respect to Shfl25875. These alternative phages complement the strains of Shigella infected by Shfl25875. For example:

1. Shfl25875 infects the majority (28 out of 29) of S. flexneri strains. The one Shfl25875-resistant strain is susceptible to Sdys9752.

2. Shfl25875 infects all (12/12 strains) S. dysenteriae type 1 strains, but shows poor infectivity (2 out of 12) against the other S. dysenteriae types (2 through 12). In contrast, Sdys9752 does not infect type 1 strains, but infects 5 of 12 type 2-12 strains. In addition, Sdys12039 infects 9 of 12 S. dysenteriae type 2-12 strains. In combination, these 3 phages infect nearly all (22/24) S. dysenteriae strains tested.

3. Shfl25875 infects 24 out of 27 S. sonnei strains tested. The three Shfl25875 resistant S. sonnei strains are susceptible to either or both of Sdys9752 and Sdys12039.

4. Shfl25875 does not infect the 5 S. boydii strains in our collection. However, Sdys9752 and Sdys12039 infects 4 out of 5 of these strains.

The Sdys9752 and Sdys12039 phages were molecularly engineered to generate reporter phages using techniques that were similar to that described for Shfl25875. In both cases, the luxAB reporter genes were inserted into non-coding regions of the genomes by homologous recombination without removing any phage DNA and thus increased the overall genome sizes by 2,108 bp. PCR analysis was used to verify the presence of luxAB in the recombinant phages, and that luxAB had integrated into the correct predicted site in the phage genome (data not shown). PCR analysis of phage lysates using primers designed to amplify a section of luxB, and to span the 5′ and 3′ junctions of luxAB integration into the phage genomes, generated the correct sized PCR products as expected. This indicated: (i) the presence of the reporter, and (ii) that the luxAB cassette had integrated at the correct genome sites as expected.

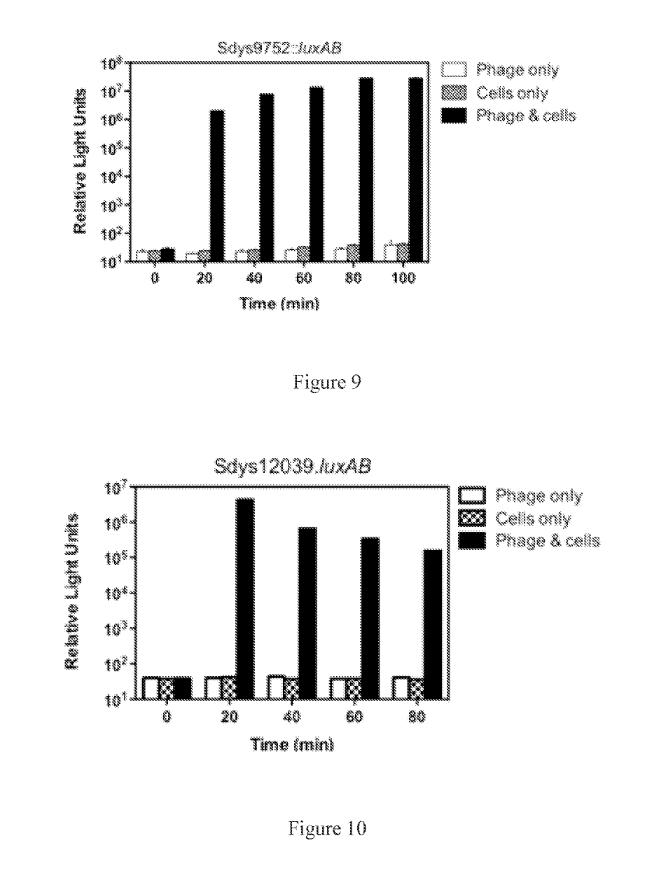

The ability of Sdys9752::luxAB and Sdys12039::luxAB to transduce a bioluminescent phenotype, and hence detect Shigella strains was analyzed (results shown in FIGS. 9-10). As shown in FIGS. 9-10, S. sonnei 9290 and S. flexneri 7-3510 were grown at 37° C. in LB broth, and mixed with Sdys9750::luxAB or Sdys12039::luxAB, respectively. Infected cultures were incubated at 37° C. and bioluminescence was measured over time following the addition of substrate n-decanal. Numbers are the mean (n=3)±SD. Reporter phage (10⁸ PFU/mL final) and cells were mixed and ‘flash’ bioluminescence was measured following the addition of the substrate decanal using a GLOMAX® 96 Microplate Luminometer (registered trademark of Promega Corporation of Madison, Wis.). Strong (10⁵-fold increase in signals over background) and rapid signals were detected 20 min after infection with both reporter phages. These data indicating that the reporters were able to infect, and express luxAB to significant levels in a very short period of time. Similar results were obtained with other Shigella strains.

The current configuration of the detection device requires the addition of an aldehyde substrate (e.g., n-decanal) in order to generate the bioluminescent signal response. In this configuration, the luciferase enzyme (encoded by luxAB) in the presence of flavin mononucleotide (naturally present in the bacterial cell), oxygen and endogenously added decanal, catalyzes the reaction resulting in light as a by-product. In another configuration, the phage is engineered to encode the genes encoding luciferase, as well as the genes encoding the fatty acid reductase complex. These latter genes (luxCDE) encode the enzymes required for making the substrate, and thus generates an inclusive detection system that does not require exogenous substrate for light production.

The previous examples discuss using the disclosed technology in a laboratory setting to test human stool samples. It is understood that one of ordinary skill in the art would be able to adapt the disclosed technology for use in other settings such as in the field to test for the presence of target bacteria such as members of the Shigella genus. It is also understood that the disclosed technology could be adapted to test other materials such as other human clinical samples (mucus, rectal swabs, and the like), drinking water, food, soil, surfaces, and the like. In other examples, the phages described herein may be included as part of a Shigella microorganism detection kit having sample(s) of the phages described herein stored in suitable container(s) packaged with one or more testing containers for collecting and testing samples to be tested for the presence of Shigella microorganism.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. A system for detecting Shigella, comprising:
a phage operable to infect a Shigella microorganism, the phage having a luxAB reporter nucleic acid configured and arranged to be expressed upon infection of the Shigella microorganism by the phage; and
a detector operable to detect expression of the luxAB reporter nucleic acid.

2. The system of claim 1, wherein the Shigella microorganism is selected from the group consisting of S. flexneri, S. dysenteriae, S. sonnei, S. boydii, and combinations thereof.

3. The detection system of claim 1, wherein the phage is selected from the group consisting of Shfl25875, Sdys9752, Sdys12039, and combinations thereof.

4. The detection system of claim 1, wherein the expression of the luxAB reporter is detected as bioluminescent light.

5. The detection system of claim 1, wherein the phage further includes a luxCDE reporter nucleic acid.

6. The detection system of claim 1, further comprising an aldehyde catalyst.

7. A kit, comprising:
a phage operable to infect Shigella microorganism, comprising a reporter configured and arranged to be expressed upon infection of the Shigella microorganism by the phage, in a suitable container; and
one or more containers to mix the phage with a test sample that may contain Shigella microorganism.

8. The kit of claim 7, further comprising an aldehyde catalyst in a suitable container.

9. The kit of claim 7, further comprising a bioluminescent detector.

10. The kit of claim 7, wherein the Shigella microorganism is selected from the group consisting of S. flexneri, S. dysenteriae, S. sonnei, S. boydii, and combinations thereof.

11. The kit of claim 7, wherein the phage is selected from the group consisting of Shfl25875, Sdys9752, Sdys12039, and combinations thereof.

12. A method of detecting the presence of Shigella microorganism, comprising:
a) providing a phage operable to infect a Shigella microorganism, the phage comprising a reporter configured and arranged to be expressed upon infection of the Shigella microorganism by the phage;
b) contacting a test sample with the phage; and
c) detecting expression of the reporter to indicate the presence of Shigella microorganism in the test sample.

13. The method of claim 12, wherein said detecting step is expressed as bioluminescent light.

14. The method of claim 12, wherein the Shigella microorganism is selected from the group consisting of S. flexneri, S. dysenteriae, S. sonnei, S. boydii, and combinations thereof.

15. The method of claim 12, wherein the phage is selected from the group consisting of Shfl25875, Sdys9752, Sdys12039, and combinations thereof.

16. The method of claim 12, wherein said contacting step further includes contacting an aldehyde catalyst with said test sample and said phage.

17. The method of claim 12, wherein the source of said test sample is selected from the group consisting of human clinical samples, water, food, soil, and combinations thereof.

18. An isolated DNA coding for a Shigella-detecting phage, said phage having the polynucleotide sequence set forth in SEQUENCE LISTING 1.

19. An isolated DNA coding for a Shigella-detecting phage, said phage having the polynucleotide sequence set forth in SEQUENCE LISTING 2.

20. An isolated DNA coding for a Shigella-detecting phage, said phage having the polynucleotide sequence set forth in SEQUENCE LISTING 3.
